(19) United States
(12) Patent Application Publication
(10) Pub. No.: US 2026/0054087 A1
(43) Pub. Date: Feb. 26, 2026

(54) COMPACT ULTRAVIOLET LIGHT DELIVERY DEVICE FOR OPHTHALMIC PROCEDURES

(71) Applicant: RxSight, Inc., Aliso Viejo, CA (US)

(72) Inventors: John Kondis, Irvine, CA (US); Ilya Goldshleger, Ladera Ranch, CA (US); Jeremy Dong, Aliso Viejo, CA (US); David Calligori, Rancho Santa Margarita, CA (US)

(73) Assignee: RxSight, Inc., Aliso Viejo, CA (US)

(21) Appl. No.: 18/813,764

(22) Filed: Aug. 23, 2024

Related U.S. Application Data
(63) Continuation of application No. 18/813,525, filed on Aug. 23, 2024.

(51) Int. Cl. A61N 5/06; A61F 2/16

(52) U.S. Cl. CPC A61N 5/06; A61F 2/16; A61F 2002/1699; A61N 2005/0661

(57) ABSTRACT

A compact UV light delivery device comprises a UV LED, integrated into the compact UV light delivery device, to generate a UV beam; a homogenizing beam coupler, to receive the UV beam from the UV LED, and to homogenize the UV beam such that a measure of non-uniformity of the output homogenized beam is smaller than the measure of non-uniformity of the received UV beam; an illumination optics, to receive the homogenized beam and to forward it as an illumination beam; a spatial light modulator, to modulate the illumination beam into a modulated beam according to a procedure profile; a projection optics, to receive and to project the modulated beam as a projection beam through its objective into an eye of a patient; and a binocular-free imaging system, to image the eye of the patient via the same objective, and to present the image on a user interface.

### CROSS REFERENCE TO RELATED APPLICATIONS

This application claims priority from and benefit of U.S. patent application Ser. No. 18/813,525 entitled: “Compact ultraviolet light delivery device for ophthalmic applications”, to J. Kondis et al., filed on Aug. 23, 2024, hereby incorporated in its entirety by reference.

### TECHNICAL FIELD

This document is related to compact UV light delivery devices, more precisely to compact ultraviolet light delivery devices for ophthalmic procedures.

### BACKGROUND

The surgical procedure performed in the largest number per year in the United States is the removal of a natural lens from the eye that has cataract, and the implantation of an Intra Ocular Lens, or IOL, in its place. This surgical procedure and the IOLs became quite sophisticated over the last few decades and in a majority of the cases they deliver reasonably good patient outcomes. However, in a substantial fraction of the cases, it has been observed that the IOLs tend to shift, move, or rotate away from their optimal location after the surgery, caused by the healing process. This IOL shift tends to cause residual refractive errors and thus a deterioration of the visual acuity of the patient and an erosion of patient satisfaction.

About 25 years ago a creative solution has been developed to address this problem. The idea of a Light Adjustable Lens, or LAL, has been put forward, whose optical properties can be modified non-surgically after the implantation to compensate for the residual refractive errors, thereby restoring the good visual acuity of the patient. Very briefly, these LALs are formed from a polymerized silicone matrix with photoreactive additives that include polymerizable macromers and photoinitiators. Several weeks after the implantation of the LAL, the patient's visual acuity and refraction is evaluated. If desired, the optical properties of the LAL can be adjusted by applying an illumination with a procedure profile, or beam shape, to the eye with a light delivery device to selectively activate photoinitiators in the LAL. The activated photoinitiators then induce a polymerization process of the macromers in a spatially varying manner according to the procedure profile. This polymerization causes a spatially varying change of the curvature, and to a lesser degree, of the index of refraction of the LAL, thus changing the refractive properties, in particular the optical power, of the LAL such that these changes compensate the residual refractive errors of the LAL. If desired, the adjustment procedure can even extend the depth of focus of the LAL. Finally, once the desired optical power, or refractive change of the LAL has been induced by the adjusting illumination, a so called “lock-in” illumination is applied at a later time, to immobilize the remaining mobile macromers, thus preventing any further change of the refractive properties of the LAL. Typically, the adjusting illuminations are performed with a beam having a lower optical power, or intensity, whereas the lock-in illuminations are performed with a beam having a higher optical power or intensity.

To avoid any refractive changes in the LAL after the lock-in, caused by the visible solar spectrum during everyday outdoor activities, the photoinitiators of the LALs were selected such that their activation wavelength was outside the visible spectrum, in the near ultraviolet (UV). The chemistry of these LALs has been described, e.g. in the U.S. Pat. No. 6,450,642, entitled: “Lenses capable of post-fabrication power modification”, to J. Jethmalani et al., hereby incorporated in its entirety by reference.

The illumination devices, also called Light Delivery Devices, or LDDs that were developed to deliver this UV illumination with the procedure profile, and the method of the illumination itself, have been described also, e.g. in U.S. Pat. No. 6,905,641, entitled “Delivery system for post-operative power adjustment of adjustable lens”, to B. Platt et al., also incorporated in its entirety by reference.

However, these LDD devices were developed more than two decades ago, and thus have not benefited from the numerous technical developments since that time. For example, these LDDs use mercury arc lamps as their UV light source, which are problematic for several reasons, as described below in more detail. Also, their optics were large and bulky. Their efficiency to convert electric power to optical power was also strikingly low. Finally, they used traditional binocular imaging systems which inflated the size of these LDDs.

Therefore, there is a need for more compact and more energy efficient light delivery devices with higher quality optics and better imaging systems to make the advantages of the light adjustable lens available to a wider range of patients.

### SUMMARY

To address the above described needs, embodiments of a compact ultraviolet (UV) light delivery device were developed that comprise an integrated UV LED, integrated into the compact UV light delivery device, to generate a UV beam; a homogenizing beam coupler, to receive the UV beam from the integrated UV LED, and to homogenize the UV beam such that a measure of non-uniformity of the output homogenized beam is smaller than the measure of non-uniformity of the received UV beam; an illumination optics, to receive the homogenized beam and to forward it as an illumination beam; a spatial light modulator, to receive and to modulate the illumination beam into a modulated beam according to a procedure profile; a projection optics, to receive and to project the modulated beam as a projection beam through its objective into an eye of a patient; and a binocular-free imaging system, to image the eye of the patient via the same objective utilizing an imaging beam splitter, and to present the image on a user interface.

### BRIEF DESCRIPTION OF THE DRAWINGS

FIGS. 1A-B show some existing light delivery devices, based on mercury lamps.

FIGS. 2A-B show schematic embodiments of the compact UV light delivery device (compact UV LDD).

FIG. 3 shows an embodiment of the compact UV LDD.

FIGS. 4A-E show several aspects and embodiments of the homogenizing beam coupler.

FIG. 5 shows a section of the compact UV LDD with the homogenizing beam coupler and the illumination optics.

FIG. 6 shows the schematic optical design of an embodiment of the compact UV LDD.

FIGS. 7A-B show the details of a spatial light modulator.

FIG. 8 shows details of the imaging system of the compact UV LDD.

FIG. 9 shows an embodiment of a digital reticle.

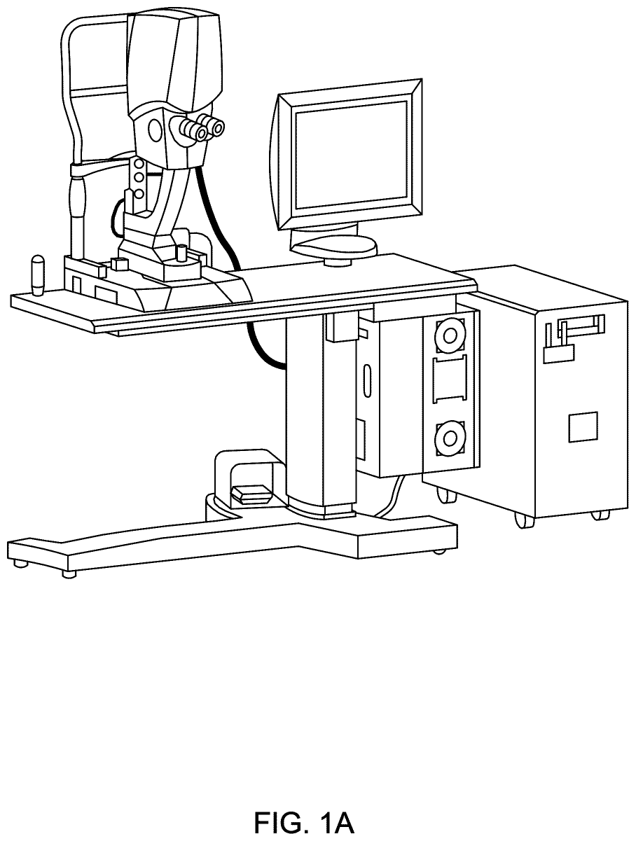

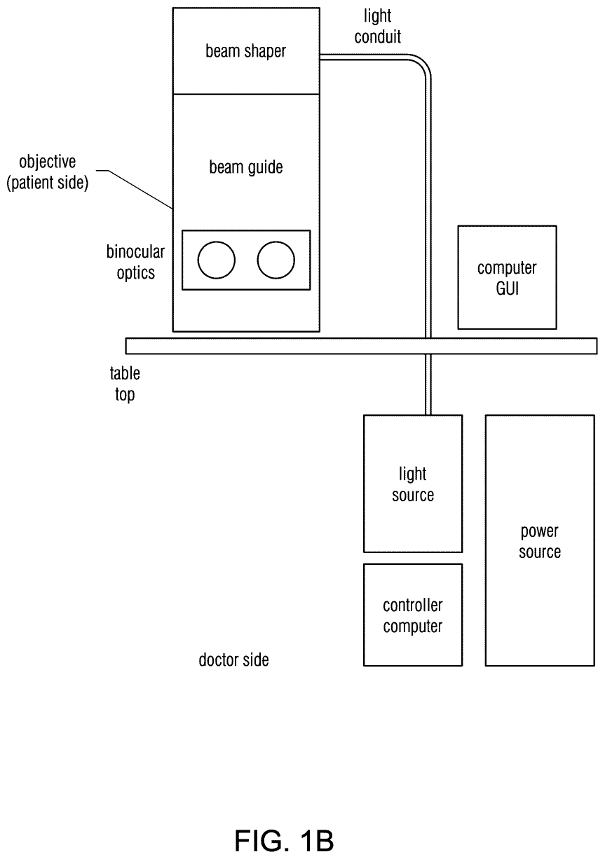

### DETAILED DESCRIPTION

In order to address the above medical and technical needs, a Compact Ultraviolet (UV) Light Delivery Device, or C-LDD, has been developed. Its design implements at least four inventive Design Concepts, each of which provides qualitative improvements over existing devices by themselves, as well as in combination.

(1) Exchange of the UV light source from a mercury lamp to a UV LED. This allows integration of the UV light source into the C-LDD and greatly reduces losses. Previous devices used a mercury arc lamp as the UV light source. Mercury lamps are capable of generating UV light with a high optical output power. However, their efficiency is low and thus they also generate a large amount of waste heat that needs to be managed. Therefore, the lamp, its cooling system, and their power supply had to be accommodated in a separate enclosure previously to avoid interfering with the optical performance of the optics of the LDD. This separation necessitated the use of a fiber bundle to transmit the UV light from the separate mercury lamp enclosure to the device optics. This fiber bundle was a second source of considerable loss. Overall, more than 95% of the electrical input power was lost and wasted as heat, and on top of this, more than 30-35% of the optical power of the generated UV beam was also lost before it reached the device optics.

To dramatically reduce these losses, the C-LDD uses a UV LED instead of the mercury lamp as its UV light source. Presently, however, UV LEDs are available only with limited optical power, in the range of 2 W-3 W. However, the lock-in of the light adjustment procedure of the LAL needs considerable optical power, also in the range of 2 W-3 W. While mercury lamps are capable of producing this high power in spite of their substantial losses, in the C-LDD that uses a UV LED, losses have to be aggressively minimized to meet the optical power requirements even marginally. Therefore, switching out the mercury lamp to the UV LED in a high optical power application like the lock-in is very counterintuitive. To minimize the losses and to deliver the required power even marginally, new technical solutions need to be introduced along the entire optical system of the C-LDD. The introduction of these quantitative differences turned into the qualitative difference of making the switching from the mercury lamp to a UV LED possible.

(2) Introduction of a multi-functional homogenizing beam coupler that performs two or three functions: homogenizes the beam, reduces the beam numerical aperture, and creates a beam shape that advantageously approximates a circle better. The UV beam, emitted by the UV LED, has notable spatial variations, caused by the structure and spatial variation of the LED die, while the UV beam output by the C-LDD needs to be as homogeneous as possible. Also, the UV LED generates the UV beam with a high numerical aperture, while the LAL adjustment procedure needs the beam to have a lower numerical aperture. Finally, the generated UV beam may have a square beam shape, caused by the typically square shape of the UV LED. However, the LAL adjustment procedure uses a circular beam shape, to match the circular shape of the LAL. Thus, a transformation of the initial square beam shape to an eventual circular beam shape is necessary, preferably with as little losses as possible. Previous devices managed these three needs by separate optical elements, each of which taking up precious space. In contrast, the C-LDD design found a way to perform at least two, possibly all three functions with a single, integrated optical element, the homogenizing beam coupler. The multi-functionality of this optical element therefore substantially reduces the size the C-LDD, lowers its losses, and increases its efficiency.

(3) Introduction of a dual prism design for the spatial light modulator. This design greatly reduces the overall size of the optics, because the lenses of the optics can be brought much closer to the spatial light modulator. In existing devices, the angular difference between the illumination beam incident on the spatial light modulator, and the modulated beam, reflected by the spatial light modulator, is small. To achieve the needed spatial separation between the pre-modulation and post-modulation beams, the pre-modulation illumination lenses and the post-modulation projection lenses had to be placed far from the spatial light modulator. This increased the size of the entire optics and required the introduction of Z folding mirrors. In contrast, the C-LDD introduced a dual prism design for the spatial light modulator, which allowed bringing the illumination lenses and the projection lenses much closer. This dual prism design reduced the total optical path by 40%, and thus eliminated the need for Z folding mirrors. All this further reduced the size of the C-LDD, and made its optics more resistant against misalignment.

(4) Elimination of the binocular imaging optics by switching to single optical channel imaging. This switch also reduces the size of the C-LDD substantially. Historically, ophthalmic devices use binocular optics for imaging, familiar for the doctors. However, binocular imaging requires a stereoscopic beam, split widely to the pupillary distance, often with a Porro prism. This makes the size of the imaging optics bulky and increases its footprint. In contrast, C-LDD devices have an imaging system that uses a single channel imaging optics, not a binocular imaging optics. Also, it is fully electronic. The elimination of the binocular optics enabled yet another way to substantially reduce the size of the C-LDD device, while at the same time it introduced all the benefits of electronic imaging. Switching to the electronic imaging introduced the multiple benefits of modern imaging techniques for the C-LDD.

The creation of the C-LDD with a size reduced by a combination of some, or all, of the above Design Concepts offers medical benefits. Beyond that, the substantially reduced size also makes the C-LDDs perform more robustly, and need less frequent service. Finally, the smaller size of the C-LDD can translate to a smaller price as well. This can make the LAL lenses affordable and thus available to a much larger number of patients, for them to enjoy their medical benefits.

In what follows, embodiments of the compact UV LDD 100 will be described that implement some of these Design Concepts, alone, or in combination with the other Concepts.

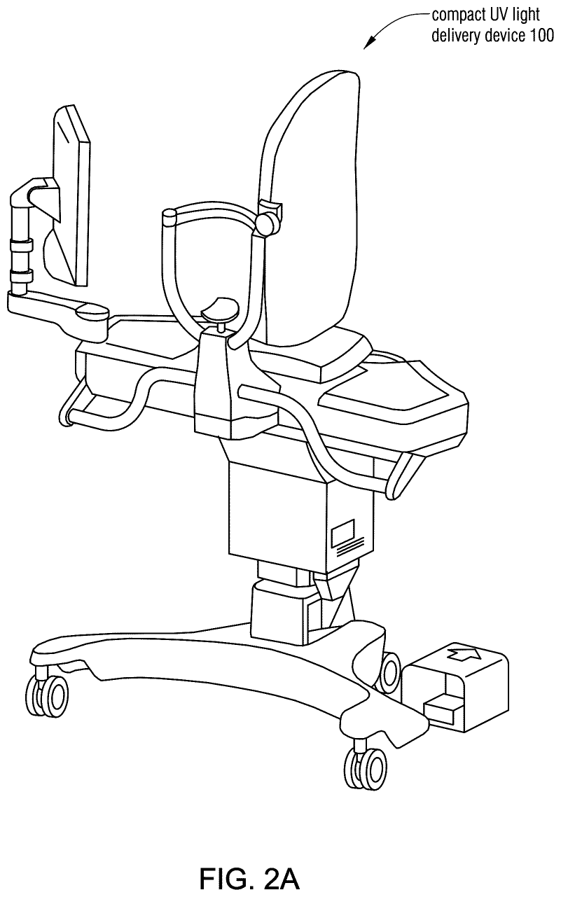

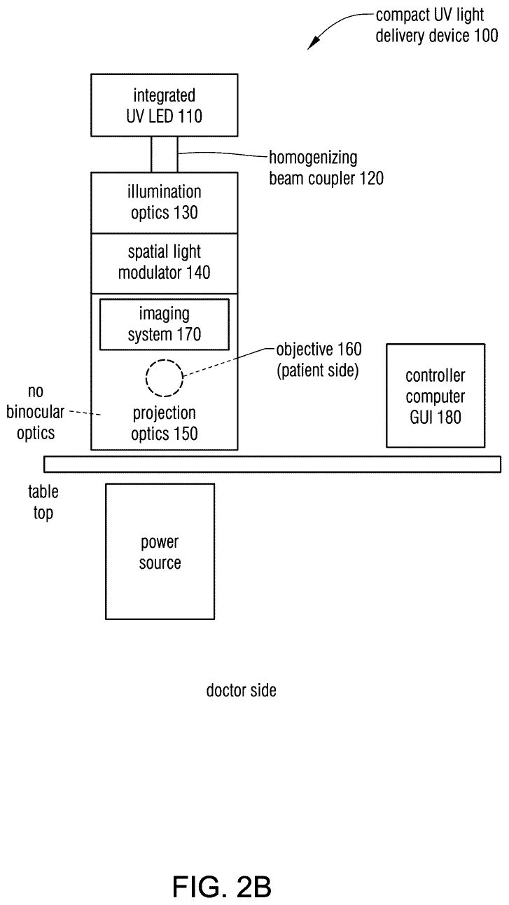

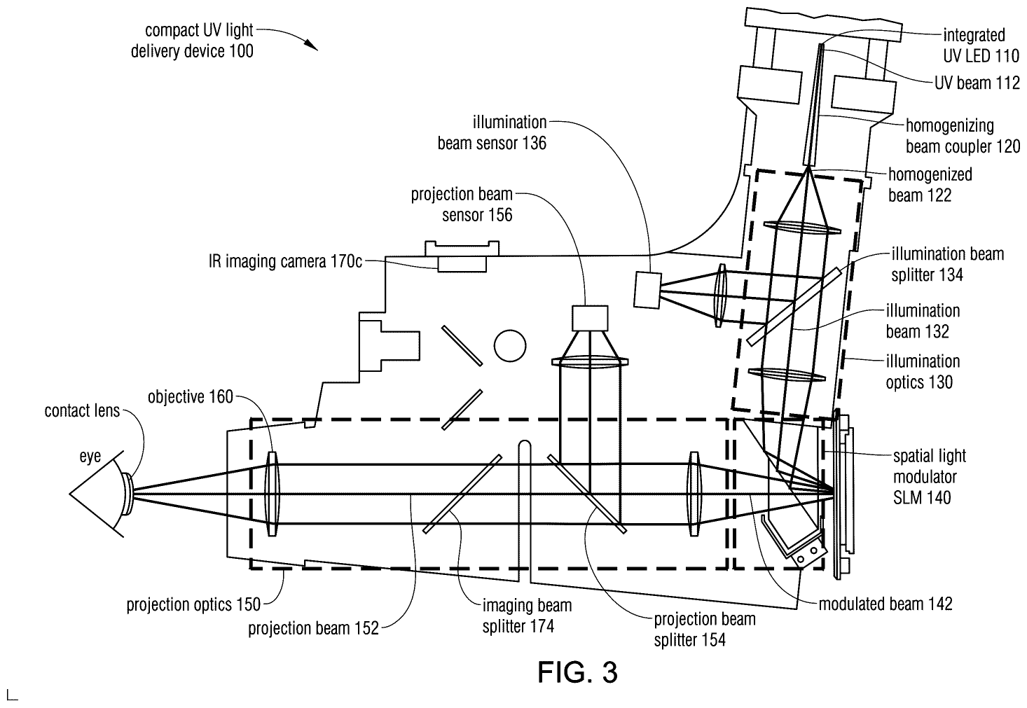

FIGS. 2A-B show schematically, and FIG. 3 shows in detail that some embodiments of a compact UV light delivery device 100, or C-LDD 100, can comprise an integrated UV LED 110, integrated into the compact UV light delivery device 100, to generate a UV beam 112; a homogenizing beam coupler 120, to receive the UV beam 112 from the integrated UV LED 110, and to homogenize the UV beam 112 such that a measure of non-uniformity of the output homogenized beam 122 is smaller than the measure of non-uniformity of the received UV beam 112; an illumination optics 130, to receive the homogenized beam 122 and to forward it as an illumination beam 132; a spatial light modulator (SLM) 140, to receive and to modulate the illumination beam 132 into a modulated beam 142 according to a procedure profile; a projection optics 150, to receive and to project the modulated beam 142 as a projection beam 152 through its objective 160 into an eye of a patient; and a binocular-free imaging system 170, to image the eye of the patient via the same objective 160 utilizing an imaging beam splitter 174, and to present the image on a user interface 180.

A design notion regarding the shown Figures. While the various optics, such as the illumination optics 130 and the projection optics 150 are shown to include two lenses each, each of these lenses can represent a group of lenses, as it is a standard practice in optical design. A lens is often replaced by a group of lenses to deliver a similar optical power, but beyond that to also deliver some additional design goals, such as reduced sensitivity to thermal expansion, reduced higher order aberrations, or reduced chromatic aberration, just to name a few. Since there are a large number of different lens groups each of which can be preferred to satisfy different design criteria, the Figures in this document will indicate the lenses, or lens groups, of differently optimized embodiments only with a single effective lens each, which lens, however, can very well reference a lens group in a particular implementation.

To contextualize the importance of the Design Concept (1), the use of the integrated UV LED 110, it is recalled that existing devices use mercury lamps. These mercury lamps are very inefficient sources of UV light. A typical mercury lamp itself operates with a voltage in the 40V-90V range, with a current in the 3 A-6 A range, thus the lamp itself typically consumes 200 W-300 W. These lamps give off a lot of heat, and thus require a powerful cooling system and a high current controller system. All in all, mercury lamp systems typically use 3 A-4 A current from a 120V AC supply, thus using up 300 W-400 W total input electrical power. On the other hand, their optical output power, i.e. the power of the output UV beam is typically only 4 W-6 W. Therefore, a Power Efficiency Ratio (PER) of such mercury lamp-based devices, defined as the optical output power divided by the electrical input power is in the 0.5%-2% range: it is very low. Accordingly, most of the electrical input power is emitted as waste heat, in the range of 300 W-400 W. This great heat production necessitates a large cooling system that itself requires additional electrical input power. To avoid problems arising from the heating of the optics of the device itself, and from the vibrations by the cooling fans, these heat-producing mercury lamps necessitate separate housing in a separate enclosure. The separate housing, in turn, requires a long and lossy light conduit, or fiber bundle to connect the mercury lamp to the optics of the device. Existing devices can lose 20%-30% of the generated UV beam power in this fiber bundle. Of course, this necessitates the generation of an even higher optical output power by the mercury lamp itself so that the optical power that arrives to the device after this loss in the fiber bundle still meets the optical power requirements of the LAL adjustment procedure. Finally, the use of mercury, a toxic substance, in this ophthalmic application also raises concerns and necessitates mitigation.

In contrast, in embodiments of the compact UV light delivery device 100, the integrated UV LED 110 is capable of operating with a voltage in a range of 2V-5V and with a current in a range of 0.1 A-2.0 A, thereby receiving an electrical input power in the 0.2 W-10 W range. Further, in embodiments the integrated UV LED 110 can be capable of outputting the UV beam 112 with an optical output power in a range of 0.1 W-3 W. The various LAL procedures require very different optical output powers: the adjustment of the Light Adjustable Lens (LAL) can require low optical powers in the range of a few mW to several hundred mW, while the lock-in of the adjustments of the LAL can require high optical powers, such as in the range of 1 W-3 W, for example in the range of 0.5 W-1.5 W. Thereby, UV LEDs 110 can operate with a maximum power efficiency ratio PER in a range of 10%-50%. It is noted that in some UV LEDs this power efficiency ratio can be even higher than 50% when the UV LED 110 is not operated at high currents. This is a much higher power efficiency ratio than that of mercury lamps and thus enables a qualitatively better design of the compact UV LDD 100. In particular, the high efficiency of the UV LEDs 110 means that their waste heat emission is very low, only a few W. This heat can be managed comfortably, in contrast to the mercury lamps. Therefore, the UV LEDs 110 can be housed within the light delivery device 100, in other words, they can be “integrated”. A consequence of housing the UV light source in an integrated manner with the optics means that there is no need for the long and lossy fiber bundle. This preempts the associated power loss of up to 30%. This also means that the optical output power of the UV LEDs 110 can be lowered from the 4 W-6 W of the mercury lamps to only about 3 W. This lower optical power reduces the needed electrical input power substantially. In sum, switching the UV light source from a mercury lamp to the UV LEDs 110 eliminates most of the electrical input power losses, and substantially reduces the optical outpower losses. The quantitative reduction of all these losses combines into a qualitative change: the switching to the UV LEDs 110 enables the integration of the UV light source into the LDD 100 itself. The qualitative change of this integration induces several design benefits.

It is also mentioned that the “mean time between failures”, or MTBF, is only a few hundred hours for mercury lamps, while it is many thousands of hours for UV LEDs 110. Thus, UV light delivery devices that use UV LEDs 110, require much less frequent service and less frequent change of their UV light source. This is another order of magnitude improvement of using UV LEDs 110 instead of mercury lamps.

In embodiments, a peak wavelength of the UV LED 110 can be chosen to overlap with the absorption spectrum peak of the photoinitiators that induce the chemical processes that lead to the refractive corrections of the LALs. Embodiments of the integrated UV LEDs 110 can be configured to generate the UV beam 112 with a peak wavelength in a range of 350 nm-400 nm, or, more broadly in a range of 300 nm-400 nm. In some cases, the peak wavelength can be in a range of 360 nm-370 nm. It reduces the losses to generate most of the output optical power of the UV beam 112 with a wavelength overlapping with the absorption spectrum peak of the photoinitiators. Part of achieving this overlap is to generate the UV beam 112 of the UV LED 110 with a narrow spectrum centered at a peak wavelength, having a “full width half maximum” less than 20 nm. In some cases, this “full width half maximum” can be less than 12 nm, or even less than 10 nm.

Finally, it is noted that in some embodiments it may be helpful to use a UV LED array instead of a single UV LED 110. However, general optical principles impose well-known limitations on when turning to a LED array leads to benefits, and when it does not.

The context of Design Concept (2), the introduction of the multi-functional homogenizer beam coupler 120, is that the size and price of the C-LDD 100 can be further reduced by making the homogenizing beam coupler 120 perform multiple functions, which were previously performed by separate optical elements and thus made the device bulkier. The homogenizing beam coupler 120 can perform any combination of the following functions. (2.1) Reducing the beam inhomogeneities caused by the uneven UV beam generation by the structure of the UV LED 110, as captured by a measure of beam non-uniformity. (2.2) Transforming the square-like beam shape, generated by the UV LED 110, into a circle-like beam shape, best suited for the LAL adjustment procedures. (2.3) Reducing the high numerical aperture, or NA, of the UV LEDs 110 so that the homogenized beam 122 has a considerably lower NA than the UV beam 112.

(2.1) One measure of a beam non-uniformity is the beam intensity root mean square (RMS) variation across the cross section of the beam. In some C-LDDs 100, the homogenizing beam coupler 120 can homogenize the UV beam 112 such that the homogenized beam 122 has a beam intensity RMS variation across the beam cross section less than 5%. In other embodiments, the homogenizing beam coupler 120 can generate a homogenized beam 122 with a beam intensity RMS variation across the beam cross section less than 3%, or less than 1%.

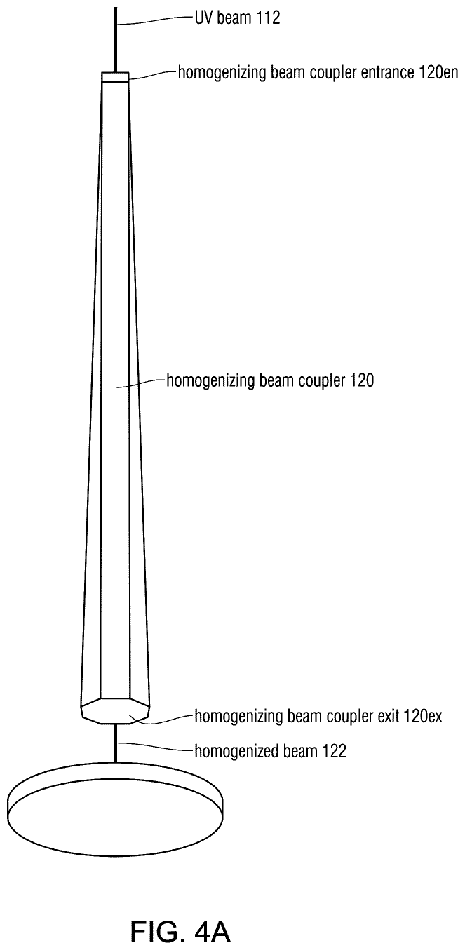

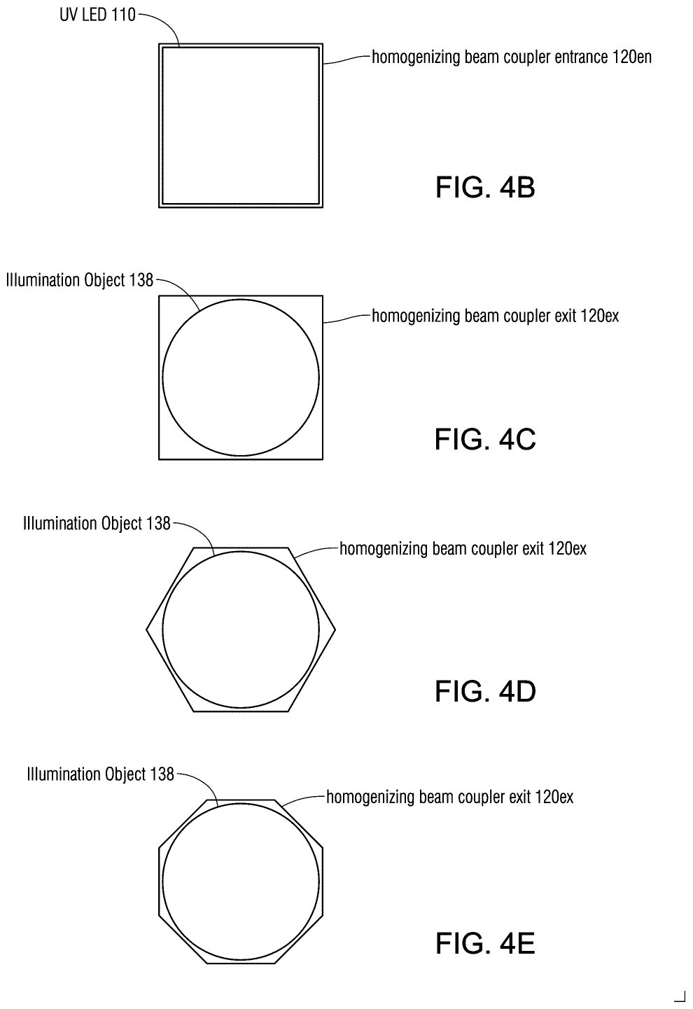

(2.2) As referred to earlier, the LAL adjustment procedure is performed with a beam having a circular beam shape, to match up with the circular shape of the LAL itself. At the same time, the procedure profile of this circular beam can still be non-circular. It can be elliptical, cylindrical, toric, or in general, different from circular in some other way in the various LAL adjustment procedures. Therefore, the optical losses caused by a potential mismatch between the exit port 120cx of the homogenizing beam coupler 120 and the entrance port of the spatial light modulator 140 can be minimized, or even avoided, by shaping the UV beam 112 from its square shape, emitted by the UV LED 110, to approach a circular shape by the time it leaves the exit port 120cx of the homogenizing beam coupler 120 as the homogenized beam 122. FIG. 4B shows that the UV LED 110 emits the UV beam 112 with a square shape. Forming the entrance 120en of the homogenizing beam coupler 120 also as a square ensures that the entire UV beam 112 enters the homogenizing beam coupler 120. This beam coupling can be made even more lossless by covering the entry port 120en of the homogenizing beam coupler 120 with an anti-reflection coating.

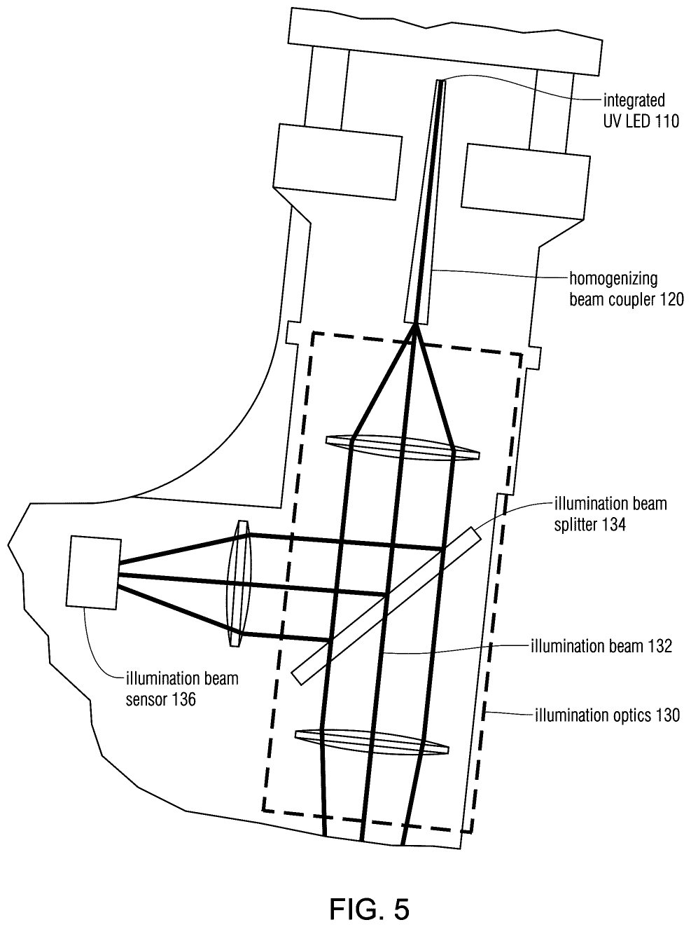

FIGS. 4C-E show that the homogenized beam 122 can be shaped to approach a more circular shape by gradually varying the cross section of the homogenizing beam coupler 120 from a square, or more generally a rectangle, entry port 120en toward a polygonal cross section having more than four corners at the exit port 120ex. FIG. 4C shows a square exit port 120cx, FIG. 4D shows a hexagonal exit port 120ex, and FIG. 4E shows an octagonal exit port 120ex, as they approach an ideal circular beam shape closer and closer. The homogenized beam 122 with this approximately circular beam shape is then imaged, or projected, onto the spatial light modulator 140 as an illumination object 138 by the illumination optics 130, as schematically indicated in FIGS. 4C-E. FIG. 5 shows an embodiment of the illumination optics 130 in some detail. A role of the illumination optics 130 is to receive the circle-approximating homogenized beam 122, and guide, or image it as the illumination beam 132 onto the spatial light modulator 140 as the illumination object 138 of FIGS. 4C-E, preferably with a circle-approximating beam shape.

(2.3) FIG. 4A shows that in some embodiments, the homogenizing beam coupler 120 can be configured to additionally reduce the numerical aperture NA of the homogenized beam 122. For example, the homogenizing beam coupler 120 can be tapered distally outward to reduce the numerical aperture NA of the homogenized beam 122, as shown. The taper increases the area of the beam cross section, thus reducing the NA. The UV LED 110 emits the UV beam 112 with an approximately isotropic angular distribution, and thus its NA is comparable to the maximum Lambertian value of 0.7-0.8. Embodiments of the homogenizing beam coupler 120 can reduce the numerical aperture of the UV beam 112 of the UV LED 110 by at least 10%. Other embodiments can reduce this NA by at least 20%. Expressed in absolute terms, embodiments of the homogenizing beam coupler 120 can reduce the numerical aperture of the UV beam 112 of the UV LED 110 so that the numerical aperture of the output homogenized beam 122 is in a range of 0.35-0.5. In yet other terms, the homogenizing beam coupler 120 can reduce the NA of the UV beam 112 by at least 0.1, 0.2, or 0.3.

Some embodiments of the homogenizing beam coupler 120 can perform all three functions: to homogenize the received UV beam 112 such that the measure of non-uniformity of the output homogenized beam 122 is smaller than the measure of non-uniformity of the received UV beam 112; to make the exit cross section of the homogenized beam 122 approximate a circle better than a square; and to generate the homogenized beam 122 with a reduced numerical aperture relative to the received UV beam 112. In some previous devices, these functions were each performed by separate elements, which took up precious space and made the entire device bulkier.

An additional aspect of the homogenizing beam coupler 120 is that it is designed to cause only a small power loss. The power loss can be defined from a ratio of an optical power of the homogenized beam 122 over the optical power of the received UV beam 112, as power loss=100% minus this power ratio. In some embodiments, this power loss can be less than 20%. In other embodiments less than 10%, or even less than 5%. From a design perspective, in some embodiments the homogenizing beam coupler 120 achieves this low power loss by being made of fused silica, which is a high quality, low loss material used in fiber optics communication. Also, its length can be relatively small, such as in the range of 1 cm-10 cm.

Returning to FIG. 5, the broad function of the illumination optics 130 is to guide, project, or image the homogenized beam 122 as an illumination beam 132 onto the spatial light modulator 140. As discussed above, it is preferable in some embodiments to image the illumination beam 132 onto the spatial light modulator 140 as an approximately circularly shaped illumination object 138. Further, the illumination optics 130 can be configured such that the numerical aperture of the output illumination beam 132 is further reduced relative to the numerical aperture of the input homogenized beam 122 by at least 20%. In some designs, for example, the NA of the UV beam 112 can be about 0.7-0.8. This can be reduced to around 0.4 by the homogenizing beam coupler 120, and then further reduced to about 0.2 by the illumination optics 130. In some designs, the illumination optics can be configured to output the illumination beam 132 with a numerical aperture in a range of 0.15-0.30. Each of these values can have a plus minus 10%-15% variation in some embodiments.

FIGS. 3 and 5 further show that in some designs the illumination optics 130 can also include an illumination beam energy sensor 136, to monitor an illumination beam energy for safety reasons, in order to prevent any damage to the eye. In embodiments, an illumination beam splitter 134 can be placed in the path of the illumination beam 132, to reflect out a portion of the illumination beam 132 toward the illumination beam sensor 136.

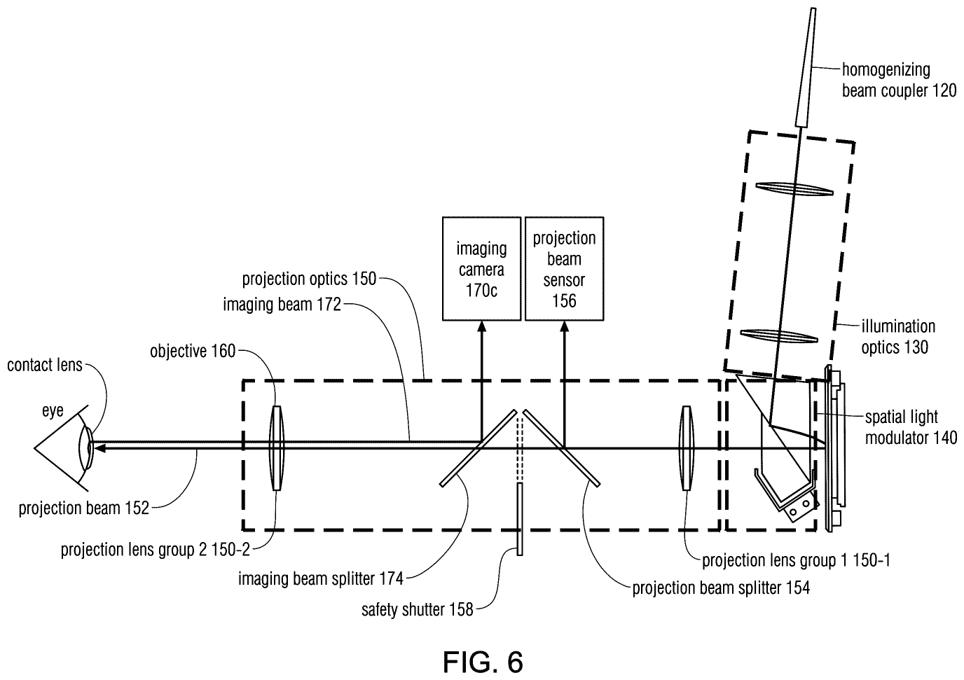

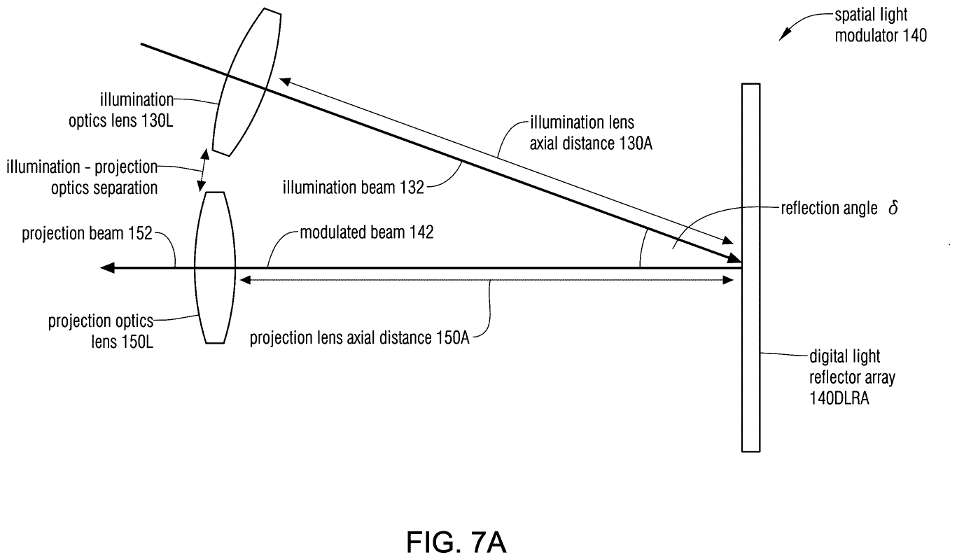

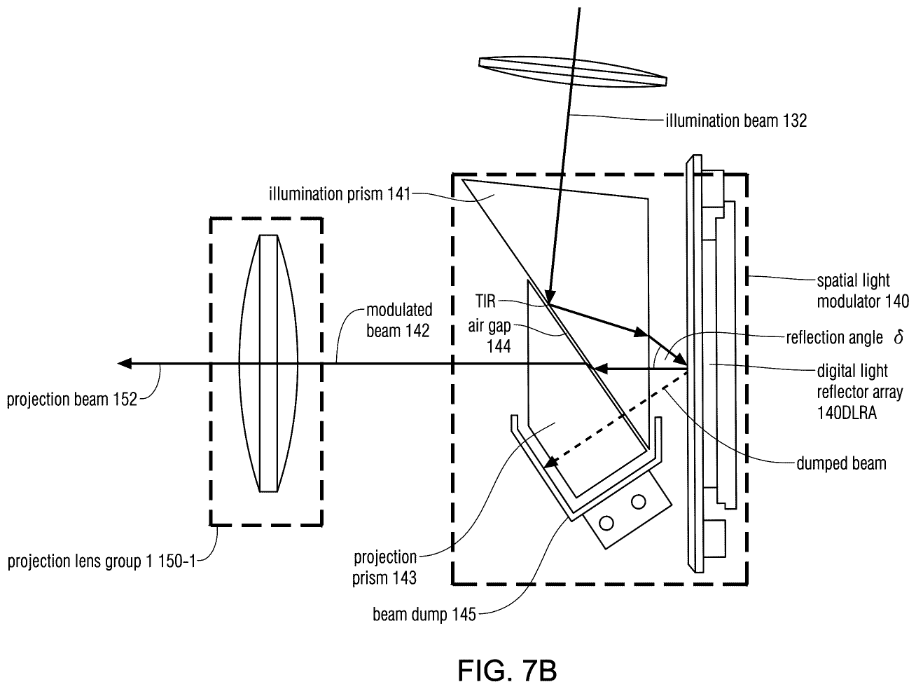

FIG. 6 shows schematically and FIGS. 7A-B show in more detail that the illumination optics 130 guides, or projects, the illumination beam 132 onto the spatial light modulator 140, that can include a digital light reflector array 140DLRA, in which individually addressable light reflectors of the array are capable of reflecting the incident illumination beam 132 by a reflection angle δ according to the procedure beam profile as the modulated beam 142, when in an “on” state. In most available digital light reflector arrays 140DLRA, this reflection angle δ is small, 2*12 degrees, because of material constraints. As before, the “procedure profile” can be a wide variety of low power beam profiles to bring about a change of the refractive properties of the Light Adjustable Lens, LAL. Subsequently, the procedure profile can also be a high power lock-in profile, to prevent any further changes in the refractive properties of the LAL. In some embodiments of the C-LLD 100, the spatial light modulator 140 can include an illumination prism 141, to redirect the illumination beam 132 toward the digital light reflector array 140DLRA with a total internal reflection (TIR) at a diagonal illumination side; and a complementary projection prism 143, with a diagonal projection side separated from the diagonal illumination side of the illumination prism 141 by a thin airgap 144, to receive the modulated beam 142 from the digital light reflector array 140DLRA through the thin airgap 144, and to forward it towards the projection optics 150.

FIG. 7B also shows that the spatial light modulator 140 can further include a beam dump 145, formed in conjunction with the projection prism 143, wherein the individually addressable light reflectors of the digital light reflector array 140DLRA are capable of reflecting the incident illumination beam 132 to the beam dump 145, when in an “off” state.

The power losses of the spatial light modulator 140 can be reduced by applying an anti-reflection coating on an entry surface of at least one of the illumination prism 141, the projection prism 143 and the digital light reflector array 140DLRA. Yet further loss reduction can be achieved by applying an anti-reflection coating on at least one of the surfaces of the airgap 144 separating the illumination prism 141 and the projection prism 143.

A note of comparison: in some existing devices, when the high-power lock-in illumination is to be generated, their version of the spatial light modulator 140 is moved out of the beam path and a highly UV-reflective mirror is moved in its place. This, of course, leads to design challenges, from the need of a motorized slider and rail system to the need of repeatedly checking and adjusting the alignment of the spatial light modulator 140 upon its return. In contrast, embodiments of the C-LDD 100 are capable of generating the modulated beam 142 for every procedure without moving the digital light reflector array 140DLRA in or out of the UV beam, or without changing a numerical aperture. Such simplified designs avoid the need for the sliding motors and the need to rechecking and possibly adjusting the alignment of the optics after every sliding in and out of the spatial light modulator 140.

FIG. 7A demonstrates Design Concept (3): a substantial reduction of the size of the C-LDD 100 by the introduction of a dual prism design for the spatial light modulator 140. In earlier devices the illumination beam 132 was directly incident on the digital light reflector array 140DLRA. Since the reflection angle δ is small, quite often about 2*12 degrees, the incoming illumination beam 132 and the outgoing modulated beam 142 are quite close to each other for an extended spatial region. Therefore, in existing devices the last illumination optics lens 130L and the first projection optics lens 150L have to be positioned quite far away from the digital light reflector array 140DLRA, at a large illumination lens axial distance 130A and a large projection lens axial distance 150L just to accommodate their spatial extent, to achieve a manageable “illumination-projection optics separation” as shown. As described below, in some relevant embodiments of the C-LDD 100, both the illumination optics 130 and the projection optics 150 are “4f optical system” at least approximately. Here f refers to the respective focal distances: that of the lens 130L for the illumination optics 130, or the focal distance of the projection optics lens 150L for the projection optics 150. For both of these “4f” optics 130 and 150, the total length of these optics 130 and 150 is four times their respective focal length. The 4f design forces these focal distances to equal the illumination lens axial distance 130A, and the projection lens axial distance 150L. This means that the size of the illumination optics 130 is equal to four times the illumination lens axial distance 130A, and the size of the projection optics 150 approximately equals four times the projection lens axial distance 150A. In short, the small deflection angle of the digital light reflector array 140DLRA forces the size of the optics 130 and 150 to be quite large, and thus the size of these existing devices quite large too. Some designs reduce this large size of the device by introducing Z folding mirrors, folding the optical path into the device. However, the quality of the imaging is quite sensitive to the precise alignment of the folding mirrors, and these folding mirrors can drift away from their precise alignment, thus necessitating repeated fine tuning and maintenance. These are all factors that negatively impact the adoption and maintenance of the existing LDD devices.

FIG. 7B shows that the dual prism Design Concept (3) minimizes, or essentially eliminates this problem by folding out the illumination beam 132 with the help of the illumination prism 141 by a large, nearly right angle. This dual prism design places the last illumination optics lens 130L and the first projection optics lens 150L far away from each other, and therefore each of them can be moved much closer to the digital light reflector array 140DLRA. This reduces their axial lens distances 130A and 150A, which in turn allows the design of the optics 130 and 150 with considerably shorter focal lengths. And finally, given the 4f designs of both the illumination optics 130 and the projection optics 150, their “4f” sizes can be substantially reduced with this dual prism design, thereby reducing the size and thus cost of the entire C-LDD 100.

In some detail, in embodiments of the C-LDD 100, the projection optics 150 can be a 4f projection system, or an approximate 4f projection system. This 4f design choice is related to the need that the projection optics 150 should be approximately telecentric both in its image space and in its object space. In its image space because the distance to the patient eye varies from patient to patient to some degree, and only telecentric beams make the beam procedure profile at the patient's eye insensitive to this distance to the patient's eye. In the object space, it is known that digital light reflector arrays 140DLRA deliver their best and most robust beam modulation with telecentric modulated beams 142.

The digital light reflector array 140DLRA can deliver the best telecentric modulated beams 142 if their incident illumination beam 132 is telecentric as well. This can be assured most directly by making the illumination optics 130 also a 4f projection system, or an approximate 4f projection system. With this design the illumination optics 130 can be approximately telecentric both in its image space and its object space.

As outlined above, this dual prism design enables the substantial reduction of the focal length of the illumination optics 130 and, importantly, the focal length of the projection optics 150. These two focal length reductions enable the substantial reduction of the overall optical path length of the C-LDD 100. In some examples of the C-LDD 100, an optical path length from the entrance of the homogenizing beam coupler 120 to the distalmost surface of the objective 160 can be less than 450 mm, in fact close to 400 nm. In contrast, this same optical path length in some existing device exceeds 600 mm. In relative terms, the optical path length of existing systems is 40%-50% longer than in embodiments of the C-LDD 100, and this largely due to the invention of the dual prism design for the spatial light modulator 140: a really impactful reduction in size, design simplicity and cost. This difference is not only quantitative, it is also qualitative. The shorter optical path length makes it possible that the entire C-LDD 100, and thus specifically its projection optics 150 does not include folding mirrors. These folding mirrors would take up space, make the device bulky and can drift out of alignment easily, thus necessitating frequent servicing of the existing devices.

Additional beneficial aspects of the projection optics 150 can be that in some embodiments, the projection optics 150 can reduce a numerical aperture of the projection beam 152 below 0.2. Such low NA values make the projection beam 152 satisfy the needs of a well-controlled LAL lens adjustment procedure.

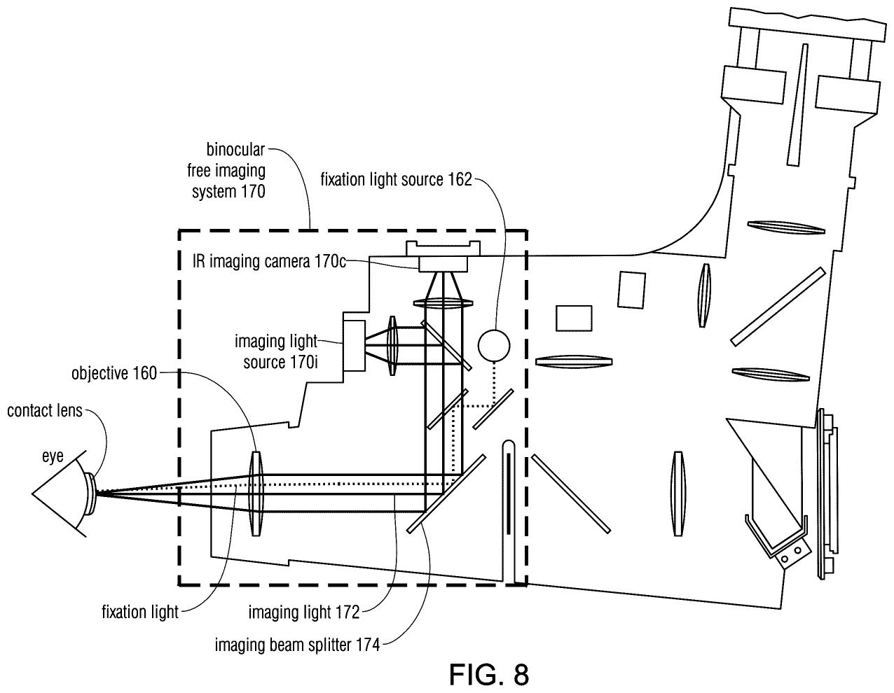

FIG. 6 and FIG. 8 show additional aspects of the projection optics 150, and additional elements of the C-LDD 100. Before proceeding, it is noted that the lenses, or lens groups, of the projection optics 150 can be called projection lens group 1 150-1, and projection lens group 2 150-2, as shown. However, it is also customary to call the last, patient-facing lens group of an optics the objective 160, which can be another name for the projection lens group 2 150-2.

In some embodiments, the projection optics 150 may include a projection beam sensor 156, to monitor a projection beam energy, and to cause a stopping of the projection beam if a projection beam energy exceeds a safety limit. As in the case of the illumination beam sensor 136, this causation can be direct, the projection beam sensor 156 sending a control signal to the UV LED 110 to shut down in case it measures the projection beam energy to exceed a safety limit. Or the causation can be indirect, the projection beam sensor 156 sending a control signal to a system controller, which then shuts down the UV LED 110. The projection sensor 156 can get a portion of the projection beam 152 deflected its way by a projection beam splitter 154, placed in the path of the projection beam 152.

In some designs, the stopping of the projection beam 152 can be caused by a safety shutter 158 blocking the projection beam 152, if the projection beam energy deviates from a safety limit. There are different ways of achieving this blocking. One of them is to rapidly slide the safety shutter 158 into the path of the projection beam 156, as shown by the dashed line in FIG. 6.

In order to help the patient to align his/her eyes with the projection beam 152 of the C-LDD 100 during a procedure, the latter may also include a fixation light source 162; and fixation light optics, to receive the fixation light from the fixation light source 162, and to forward the fixation light to the objective 160, as shown. The fixation light is often a low power green, or red, light beam, and the patient is instructed to look straight at the fixation light. Doing so reduces eye movement and helps maintain a good alignment of the projection beam 152 with the eye of the patient during the adjustment or the lock-in procedure. The fixation light optics typically involves beamsplitters that couple the fixation light into the projection optics 150.

FIG. 8 further shows that in embodiments of the C-LDD 100, the binocular-free imaging system 170 can include an infrared imaging light source 170i, to create an infrared (IR) imaging light; an infrared imaging camera 170c, to form an image from a reflected IR imaging light from the eye and to present it on a user interface; and an imaging light beam splitter 174, to couple the IR imaging light into the projection optics 150, and to couple a reflected IR imaging light out from the projection optics 150, toward the IR imaging camera 170c.

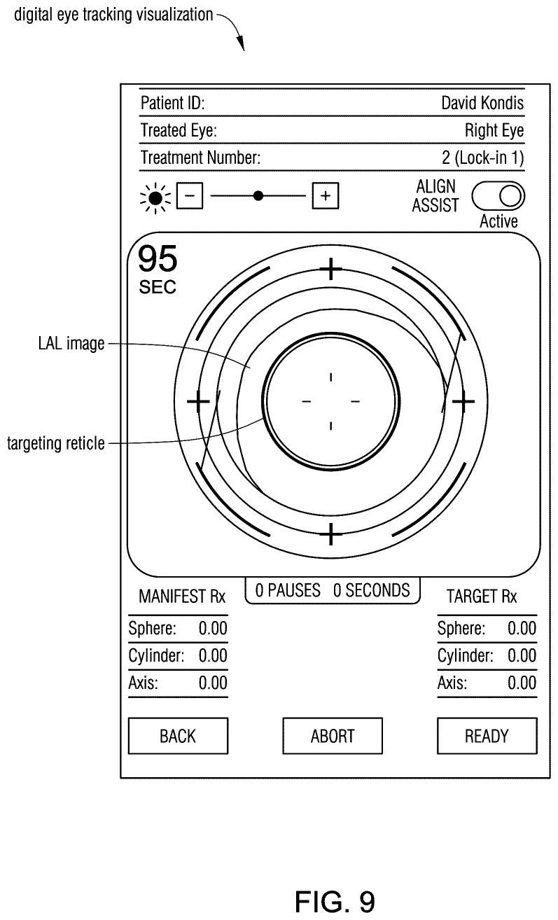

FIG. 9 shows that the binocular free imaging system 170 can include an electronic reticle, to assist the targeting of the projection beam 152 onto the eye. Several embodiments of this reticle, the associated targeting systems and the eye tracking systems based on sophisticated edge recognition and image recognition systems, and finally tracking-based illumination control systems have been previously described in the U.S. Pat. No. 11,013,593, entitled “Light Adjustable Lens Tracking System and Method”, to J. Kondis et al; and in the U.S. Pat. No. 10,932,864, entitled “Tracking-based Illumination Control System”, to J. Kondis et al, both patents incorporated hereby in their entirety by reference. Given the extensive description of the operation and benefits of these all-electronic imaging systems in the incorporated patents, there is no need to repeat them here.

One additional aspect is noted: the here-described binocular-free imaging system 170 is capable of forming the image from the reflected IR imaging light from the eye and to present it on the user interface even when a safety shutter 158 is closed. This is yet another benefit over existing systems, as in devices where the imaging system involves an actual, physical reticle, when the shutter is closed as a default, unless a procedure has been started, the eye and the targeted LAL may not be visible, and challenging solutions are needed to help the doctor to align the eye with the device.

While this document contains many specifics, details and numerical ranges, these should not be construed as limitations of the scope of the invention and of the claims, but, rather, as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to another subcombination or a variation of a subcombinations.

1. A compact UV light delivery device, comprising:
an integrated UV LED, to generate a UV beam;
a homogenizing beam coupler,
to receive the UV beam from the integrated UV LED, and
to homogenize the UV beam;
an illumination optics, to receive the homogenized beam and to forward it as an illumination beam;
a spatial light modulator,
to receive the illumination beam through a dual prism, and
to modulate the illumination beam into a modulated beam according to a procedure beam profile;
a projection optics, to receive and to project the modulated beam as a projection beam through its objective into an eye of a patient; and
an imaging system,
to image the eye of the patient via the objective utilizing an imaging beam splitter, and
to present the image on a user interface.

2. The compact UV light delivery device of claim 1, wherein:
the integrated UV LED is configured to generate the UV beam with a peak wavelength in a range of 350 nm-400 nm.

3. The compact UV light delivery device of claim 1, wherein:
the homogenizing beam coupler is configured to generate the homogenized beam with a beam intensity root mean square (RMS) variation less than 5%, wherein the beam intensity RMS variation is a measure of beam non-uniformity.

4. The compact UV light delivery device of claim 1, wherein:
the homogenizing beam coupler has an entrance port with a square or rectangle cross section, and an exit port with a polygonal cross section, having more than four corners.

5. The compact UV light delivery device of claim 1, wherein:
the homogenizing beam coupler is configured to additionally reduce a numerical aperture of the homogenized beam.

6. The compact UV light delivery device of claim 1, wherein:
the illumination optics is configured to guide the illumination beam onto the spatial light modulator with an approximately circular beam shape.

7. The compact UV light delivery device of claim 1, the spatial light modulator comprising:
a digital light reflector array, wherein individually addressable light reflectors of the array are capable of reflecting the incident illumination beam by a reflection angle according to the procedure beam profile, when in an “on” state.

8. The compact UV light delivery device of claim 7, the dual prism comprising:
an illumination prism, to redirect the illumination beam toward the digital light reflector array with a total internal reflection at a diagonal illumination side; and
a complementary projection prism, with a diagonal projection side, separated from the diagonal illumination side of the illumination prism by an airgap, to receive the modulated beam from the digital light reflector array through the airgap, and to forward it towards the projection optics.

9. The compact UV light delivery device of claim 8, the spatial light modulator comprising:
a beam dump, formed in conjunction with the projection prism, wherein
the individually addressable light reflectors of the array are capable of reflecting the incident illumination beam to the beam dump, when in an “off” state.

10. The compact UV light delivery device of claim 8, wherein:
at least one of the illumination prism, the projection prism and the digital light reflector array has an anti-reflection coating on an entry surface.

11. The compact UV light delivery device of claim 8, wherein:
at least one of the surfaces of the airgap separating the illumination prism and the projection prism has an anti-reflection coating.

12. The compact UV light delivery device of claim 1, wherein:
the projection optics is a 4f projection system, or an approximate 4f projection system.

13. The compact UV light delivery device of claim 12, wherein:
the projection optics is approximately telecentric both in its image space and its object space.

14. The compact UV light delivery device of claim 12, wherein:
the projection optics does not include folding mirrors.

15. The compact UV light delivery device of claim 12, wherein:
the illumination optics is a 4f projection system, or an approximate 4f projection system.

16. The compact UV light delivery device of claim 15, wherein:
the illumination optics is approximately telecentric both in its image space and its object space.

17. The compact UV light delivery device of claim 12, wherein:
an optical path length from an entrance of the homogenizing beam coupler to a distalmost surface of the objective is less than 450 mm.

18. The compact UV light delivery device of claim 1, wherein:
the projection optics reduces a numerical aperture of the projection beam below 0.2.
